(19) United States
(12) Patent Application Publication
(10) Pub. No.: US 2020/0197358 A1
(43) Pub. Date: Jun. 25, 2020

(54) CANNABINOID FORMULATIONS AND PHARMACEUTICAL COMPOSITIONS

(71) Applicant: Tilray, Inc., Seattle, WA (US)

(72) Inventors: Caleb Joshua EADES, Nanaimo (CA); Harpreet KAUR, Nanaimo (CA)

(21) Appl. No.: 16/721,165

(22) Filed: Dec. 19, 2019

Related U.S. Application Data
(60) Provisional application No. 62/802,377, filed on Feb. 7, 2019.
(60) Provisional application No. 62/781,832, filed on Dec. 19, 2018.

(51) Int. Cl. A61K 31/352; A61K 31/05; A61K 9/107; A61K 47/10; A61K 47/44; A61K 47/22

(52) U.S. Cl. CPC A61K 31/352; A61K 31/05; A61K 47/22; A61K 47/10; A61K 47/44; A61K 9/1075

(57) ABSTRACT

A formulation containing a cannabinoid or cannabinoid analogue in a delivery system, wherein the delivery system includes a mixture of at least one lipid and at least one surfactant, wherein the formulation self-emulsifies in an aqueous medium.

### CROSS-REFERENCES TO RELATED APPLICATIONS

This application claims the benefit under 35 U.S.C. 119 (e) of, and priority to, U.S. Provisional Patent Application No. 62/781,832, filed Dec. 19, 2018, and U.S. Provisional Patent Application No. 62/802,377, filed Feb. 7, 2019, the entire contents of these applications are hereby explicitly incorporated by reference in their entirety.

### FIELD OF THE INVENTION

The present application discloses cannabinoid formulations exhibiting improved stability, solubility and bioavailability. Pharmaceutical compositions comprising the formulations and methods for using the compositions and formulations are also disclosed herein.

### BACKGROUND OF THE INVENTION

Cannabinoids are natural extracts from the plant Cannabis sativa. The plant contains over hundred different compounds, but research has focused more on delta-9-tetrahydrocannabinol [THC] and cannabidiol [CBD]. THC is known to cause psychoactive effects or the ‘high’ felt from cannabis. THC has proven beneficial in patients suffering from Post-Traumatic Stress Disorder [PTSD], as an appetite stimulant for patients with HIV/AIDS, in reducing nausea and vomiting in patients on chemotherapy. On the other hand, CBD lacks nearly any psychoactive effect and has shown promise in treating epilepsy, including a severe form of epilepsy in children called Dravet's syndrome. CBD has also been used successfully by patients with genetic brain disorders, Crohn's disease and ulcerative colitis, and Parkinson's disease. The most common modes of administration currently used are smoking or vaporizing the plant and plant extracts; long term exposure through these routes results in lung damage and other adverse events making it highly inefficient. Oral route of drug delivery is convenient, least invasive and usually the most preferred route of administration. However, CBD and THC are highly lipophilic and undergo metabolism making them difficult to deliver via oral dosage forms.

### SUMMARY OF THE INVENTION

In accordance with one aspect, the present application is directed to a formulation containing a cannabinoid or cannabinoid analogue in a delivery system, wherein the delivery system includes a mixture of at least one lipid and at least one surfactant, wherein the formulation self-emulsifies in an aqueous medium to produce a plurality of particles having a mean particle size of about 1 to about 150 nm.

In accordance with another aspect, the cannabinoid or cannabinoid analogue includes at least one of delta-9-tetrahydrocannabinol [THC] and cannabidiol [CBD].

In accordance with certain aspects, the delivery system further includes an osmotic agent.

The present invention is also directed to pharmaceutical compositions containing the cannabinoid or cannabinoid analogue formulation disclosed herein. In some cases, the pharmaceutical composition may be in the form of a liquid dosage form, a solid dosage form, a capsule, a semi-solid, an orodispersible film, beverage, food or orodispersible tablet.

In accordance with certain aspects, the pharmaceutical composition is stable for a period of at least six months at room temperature as measured in accordance with ICH Guidance.

The present application is also directed to a method of reducing side effects associated with chemotherapy or radiation treatment, alleviating pain or suppressing appetite in a subject in need thereof by administering to the subject the pharmaceutical composition disclosed herein.

### BRIEF DESCRIPTION OF THE DRAWINGS

FIG. 1 is a graph showing CBD potency in capsules for four formulae in accordance with certain embodiments at two storage conditions over a period of 9 weeks.

FIG. 2 is a graph showing THC potency in capsules for four formulae in accordance with certain embodiments at two storage conditions over a period of 9 weeks.

FIG. 3 is a graph showing the dissolution profile for different exemplary formulations in water.

FIG. 4 is a graph showing the dissolution profile for different exemplary formulations in water.

FIG. 5 is a graph showing the dissolution profile for different exemplary formulations in simulated gastric fluid.

FIG. 6 is a graph showing the dissolution profile for different exemplary formulations in simulated gastric fluid.

FIG. 7 is a graph showing the dissolution profile for different exemplary formulations in intestinal gastric fluid.

FIG. 8 is a graph showing the dissolution profile for different exemplary formulations in simulated intestinal fluid.

### DETAILED DESCRIPTION OF THE INVENTION

### Definitions

The following are definitions of terms used in the present specification.

The term “effective amount” or “therapeutically effective amount” refers to that amount of a compound or pharmaceutical composition described herein that is sufficient for the intended application including, but not limited to, disease treatment, as illustrated below. The therapeutically effective amount can vary depending upon the intended application (in vitro or in vivo), or the subject and disease condition being treated, e.g., the weight and age of the subject, the severity of the disease condition, the manner of administration and the like, which can readily be determined by one of ordinary skill in the art. The term also applies to a dose that will induce a particular response in target cells. The specific dose will vary depending on, for example, the particular compounds chosen, the dosing regimen to be followed, whether it is administered in combination with other agents, timing of administration, the tissue to which it is administered, and the physical delivery system in which it is carried.

As used herein, the terms “treatment”, “treating”, “palliating” and “ameliorating” are used interchangeably. These terms refer to an approach for obtaining beneficial or desired results including, but not limited to, therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient can still be afflicted with the underlying disorder. For prophylactic benefit, the pharmaceutical compositions can be administered to a patient at risk of developing a particular disease, or to a patient reporting one or more of the physiological symptoms of a disease, even though a diagnosis of this disease may not have been made.

A “therapeutic effect,” as that term is used herein, encompasses a therapeutic benefit and/or a prophylactic benefit as described above. A prophylactic effect includes delaying or eliminating the appearance of a disease or condition, delaying or eliminating the onset of symptoms of a disease or condition, slowing, halting, or reversing the progression of a disease or condition, or any combination thereof.

In certain embodiments, the invention also relates to the use of a compound according to the invention, for the manufacture of a medicament for the treatment or prevention of any one of the diseases or conditions disclosed herein.

In view of the utility of the compounds according to the invention, there is provided a method of treating warm-blooded animals, including humans, suffering from any one of the diseases or conditions mentioned herein, and a method of preventing in warm-blooded animals, including humans, any one of the diseases or conditions mentioned herein.

The methods comprise the administration, preferably oral administration, of a therapeutically effective amount of a compound according to the invention to warm-blooded animals, including humans.

Therefore, the invention also relates to a method for the prevention and/or treatment of any one of the diseases or conditions mentioned herein comprising administering a therapeutically effective amount of compound according to the invention to a patient in need thereof.

The formulations described herein can be used alone, in combination or in combination with other pharmaceutical agents such as other agents used in the treatment of various conditions. In such combinations, the compounds of the present invention may be utilized in combination with one or more other drugs in the treatment, prevention, control, amelioration, or reduction of risk of diseases or conditions for which compounds disclosed herein or the other drugs may have utility, where the combination of the drugs together are safer or more effective than either drug alone.

One skilled in the art will recognize that a therapeutically effective amount of the compounds of the present invention is the amount sufficient to bring about the intended effect and that this amount varies inter alia, depending on the type of disease, the concentration of the compound in the therapeutic formulation, and the condition of the patient. Generally, an amount of the compounds disclosed herein to be administered as a therapeutic agent for treating diseases and other conditions, such as the disorders described herein, may be determined on a case by case by an attending physician or other practitioner.

The amount of a compound according to the present invention, also referred to here as the active ingredient(s), which is required to achieve a therapeutic effect may vary on case-by-case basis, with the particular compound, the route of administration, the age and condition of the recipient, and the particular disorder or disease being treated. A method of treatment may also include administering the active ingredient on a regimen of between one and four intakes per day. In these methods of treatment, the compounds according to the invention are preferably formulated prior to admission. As described herein below, suitable pharmaceutical formulations are prepared by known procedures using well known and readily available ingredients.

In accordance with one aspect, the present application provides formulations containing a cannabinoid or cannabinoid analogue in a delivery system, wherein the delivery system includes a mixture of at least one lipid and at least one surfactant, wherein the formulation self-emulsifies in an aqueous medium to produce a plurality of particles having a mean particle size of about 1 to about 150 nm, more particularly about 1 to about 50 nm, about 1 to 25 nm, and in some cases about 1 to 10 nm. In other cases, the particles have a mean particle size of about 10 to about 150 nm, more particularly about 25 to about 100 nm, and in some cases about 50 to 150 nm.

The term “analog” refers to a compound that is structurally related to naturally occurring cannabinoids, but whose chemical and biological properties may differ from naturally occurring cannabinoids. In the present context, analog or analogs refer to compounds that may not exhibit one or more unwanted side effects of a naturally occurring cannabinoid. Analog also refers to a compound that is derived from a naturally occurring cannabinoid by chemical, biological or a semi-synthetic transformation of the naturally occurring cannabinoid. Examples of these compounds include, but are not limited to, cannabinol, cannabidiol, Δ9-tetrahydrocannabinol, Δ8-tetrahydrocannabinol, 11-hydroxy-tetrahydrocannabinol, 11-hydroxy-Δ9-tetrahydrocannabinol, levonantradol, Δ-11-tetrahydrocannabinol, tetrahydrocannabivarin, dronabinol, amandamide, and nabilone. Moreover, any combination of two or more of the above mentioned cannabinoids can be present in the disclosed formulations. In accordance with certain aspects, the cannabinoid or cannabinoid analogue comprises at least one of delta-9-tetrahydrocannabinol [THC] and cannabidiol [CBD].

The cannabinoid or cannabinoid analogue may be present in the formulation or other composition in an amount of from about 1 to 60, more particularly from about 2.5 to 50, still more particularly from about 5 to 40, and in certain cases from about 10 to 30, based on weight.

The delivery system may include a mixture of at least one lipid and at least one surfactant and, optionally, co-surfactant(s) or co-solvent(s), wherein the combination of the various components in certain amounts provides for a formulation that self-emulsifies in an aqueous medium.

Lipids that are useful in the present formulation include those that can solubilize or assist in solubilizing the active(s) or provide a stabilizing effect. Examples of suitable lipids include, but are not limited to, medium-chain glycerides, a long-chain glycerides, a propylene glycol fatty acid ester and mixtures thereof.

Medium-chain glycerides (MCGs) contain 6-12 carbon fatty acid esters of glycerol and may be a mono-, di- or triglyceride. Particularly useful MCGs include medium chain triglycerides. Other useful MCGs include caprylic and capric mono- and diglycerides, and blends thereof, including glyceryl monocaprylate, glyceryl dicaprylate, glyceryl monocaprate and glyceryl dicaprate. Other MCGs include caprylic/capric triglycerides, glycerol esters of lauric acid, such as glyceryl monolaurate, glyceryl dilaurate and glycerol trilaurate, and polyglycerol esters of caprylic acid.

Long-chain glycerides (LCGs) contain 14-22 carbon fatty acid esters of glycerol. The LCG may be a mono-, di- or triglyceride. Examples of LCGs include glyceryl behenate, glyceryl monolinoleate, glycerol monooleate, glycerol monostearate, glycerol monopalmitate, glyceryl dilinoleate, glycerol diooleate, glycerol distearate, glycerol dipalmitate, glyceryl trilinoleate, glyceryl triolein, glyceryl tristearate, glyceryl tripalmitate. Other examples of LCGs include simple oils including, but not limited to the following: jojoba oil, almond oil, canola oil, castor oil, cod liver oil, corn oil, cottonseed oil, evening primrose oil, fish oil, grape seed oil, olive oil, palm kernel oil, palm oil, peanut oil, rapeseed oil, safflower oil, sesame oil, soybean oil, sunflower oil, hydrogenated castor oil, hydrogenated coconut oil, hydrogenated cottonseed oil, hydrogenated palm oil, hydrogenated soybean oil, partially hydrogenated soybean oil and hydrogenated vegetable oil.

Examples of propylene glycol fatty acid esters that may be used in the formulation as the lipid component include propylene glycol monocaprylate, propylene glycol dicaprate, propylene glycol monolaurate, propylene glycol dilaurate, and propylene glycol heptanoate. These propylene glycol fatty acid esters may also be used in the formulations as surfactants when a more lipophilic excipient is included.

When targeting the development of a lipid solution, particularly useful lipid components in the present invention include those that can dissolve the compound at concentrations greater than 100 mg active per gram of excipient (“mg/g” hereinafter), and more particularly, above 150 mg/g, and still more particularly, above 500 mg/g, which may be determined via solubility assays. For cannabinoids, particularly useful lipid components include medium-chain triglycerides, phospholipids, phospholipid derivatives, vitamin E derivatives, glyceryl dibehanate, behenoyl polyoxyl-8-glycerides, Geloil SC (soybean oil glyceryl palmitostearate), glyceryl monostearate PEG-75 stearate and combinations thereof.

The surfactant has a capacity to emulsify the lipid component of the formulation and has a hydrophilic-lipophilic balance (“HLB”) of at least 1, more particularly at least 18. In some cases, the HLB for the surfactants in the formulation is between 10 and 16. The hydrophilic-lipophilic balance of a surfactant is a measure of the degree to which it is hydrophilic or lipophilic, determined by calculating values for the different regions of the molecule. An HLB value of 0 corresponds to a completely lipophilic/hydrophobic molecule, and a value of 20 corresponds to a completely hydrophilic/lipophobic molecule. HLB values for various surfactants are well known in the art.

In accordance with certain embodiments, the surfactant may be selected from propylene glycol mono- or diesters of 8-22 carbon fatty acids, sorbitan fatty acid esters including sorbitan monolaurate; polyoxyethylene sorbitan fatty acid esters such as polysorbate 20, polysorbate 40, polysorbate 60, and polysorbate 80, polysorbate 85; polyoxyethylated mono- and di-fatty acid esters such as esters of castor oil (Kolliphor® EL), hydrogenated castor oil (Kolliphor® RH40), hydroxystearic acid (Kolliphor® HS-15); glycerol macrogolglycerides such as Labrasol®, Gelucire® 44/14, Gelucire® 50/13, Labrafil®; DL-a-tocopheryl polyethylene glycol succinate; polyoxyethylene-polyoxypropylene copolymers such as poloxamer 124, poloxamer 188, poloxamer 407; polyglycerol esters of fatty acids such as polyglycerol-6-caprylate, polyglycerol-3-oleate; and ethoxylated fatty alcohols such as the Brij® surfactants.

Particularly useful surfactants in the present formulation are those with a capacity to emulsify the lipid component of the formulation, namely those surfactants, particularly non-ionic surfactants, with a HLB greater than 8 for example Span 20 and polysorbate 85 (Tween 85), or, more particularly, a HLB greater than 12, for example, Kolliphor® RH40 (also known as Macrogolglycerol hydroxystearate, PEG-40 castor oil, Polyoxyl 40 hydrogenated castor oil), Kolliphor® EL (also known as Macrogolglycerol ricinoleate, PEG-35 castor oil, Polyoxyl 35 hydrogenated castor oil, Polyoxyl-35 castor oil), Kolliphor® HS 15 (also known as Macrogol (15)-hydroxystearate, Polyethylene glycol (15)-hydroxystearate, Polyoxyethylated 12-hydroxystearic acid, Solutol® HS 15), polysorbate 20 (also known as polyethylene glycol sorbitan monolaurate, polyoxyethylene sorbitan monolaurate, TWEEN® 20), polysorbate 60 (also known as Polyethylene glycol sorbitan monostearate, Polyoxyethylene sorbitan monostearate, TWEEN® 60), polysorbate 80 (also known as Polyoxyethylenesorbitan monooleate, TWEEN® 80), Gelucire® 44/14 (Lauroyl Polyoxyl-32 glycerides), Gelucire® 48/16 (polyethylene glycol monostearate, PEG-32 stearate, polyoxylethylene stearates), Labrasol® (Caprylocaproyl Polyoxyl-8 glycerides), Gelucire® 50/13 (Stearoyl polyoxyl-32 glycerides, Stearoyl polyoxylglycerides NF, Stearoyl macrogolglycerides EP) or Vitamin E TPGS DL-a-tocopheryl polyethylene glycol succinate. In one embodiment, the surfactant may be one or more non-ionic surfactants. In addition to those non-ionic surfactants previously disclosed, additional examples of non-ionic surfactants that may be used in certain embodiments include, but are not limited to, polyoxyethylated mono- and di-fatty acid esters of castor oil or hydrogenated castor oil, and polyethylene glycol ester of caprylic/capric glycerides, and sorbitan monolaurate, and blends thereof. It is also possible to utilize a single surfactant or a combination of lipophilic and hydrophilic surfactants in a cannabinoid lipid formulation as disclosed herein. Examples of particularly useful surfactants include polyethoxylated castor oil, such as polyoxyl 35 castor oil, poloxamers, hydrogenated castor oil ethoxylates, polyoxylethylene stearates, polyoxyl glycerides, glycol monolaureate, polyglyceryl dioleate and combinations thereof. Kolliphor® EL and Gelucire® 48/16 are particularly useful. Kolliphor® EL is a nonionic solubilizer and emulsifier made by reacting castor oil with ethylene oxide in a molar ratio of 1:35. Kolliphor® EL includes glycerol polyethylene glycol ricinoleate with fatty acid esters of polyethylene glycol and free polyethylene glycols and ethoxylated glycerol. Gelucire® 48/16 contains PEG-32 (MW 1500) esters of palmitic (C16) and stearic (C18) acids. In accordance with certain embodiments, the formulation is free of cationic and/or anionic surfactants.

The amount of lipid and surfactant in the lipid-based formulation are chosen so as to enable relatively high compound loadings of cannabinoid with acceptable formulation dispersibility. In general, the formulation or composition contains between 1-90% w/w, 5-75% w/w, typically 6-70% w/w, typically 7-65% w/w, typically 8-60% w/w, lipid component and 0.01-90%, more particularly 0.1 to 45% w/w, typically 10 to 25%, 5 to 30%, 5 to 50% w/w non-ionic surfactant. In general, the ratio of lipid component to surfactant is at least 0.1:1. The ratio may be at least 0.1:1, may be at least 1:1, may be at least 1.5:1, may be at least 2:1, or may be at least 3:1. Typically useful ranges include ratios of lipid component to surfactant of about 0.1:1 to about 5:1, more particularly about 1:1 to about 1:2 and in some cases about 0.1:1 to about 1:10.

The delivery system may contain other optional excipients or other components. These optional excipients or other components may be included to provide various benefits such as improving emulsification of the lipid component in the formulation and overall drug solubility. Examples of optional excipients or other components may include phospholipids, free fatty acids, fatty acid alcohols or synthetic fatty acid derivatives including isopropyl myristate and isopropyl palmitate. Isopropyl myristate and isopropyl palmitate may also be added to the lipid formulation as a cosolvent, for the purpose of improving drug solubility in the formulation. Other example cosolvents may include propylene glycol, polyethylene glycol, triacetin, glycerol, ethanol and diethylene glycol monoethyl ether, or other pharmaceutically acceptable cosolvents.

In accordance with certain aspects, the formulation may also include an osmotic agent. One example of a useful osmotic agent is polyethylene glycol (PEG). In accordance with certain aspects, the PEG has an average molecular weight (for example a weight average molecular weight), in Daltons, within the range 2,000 to 10,000, preferably 2,500 to 8,500, preferably 3,000 to 8,000, more preferably 3,000 to 6,000, more preferably 2,500 to 6,500, more preferably 2,500 to 4,500 for example 3,000 to 4,500, for example 3,000 to 4,100, for example 3,000 to 4,000. The PEG may have an average molecular weight within the range 6,000 to 10,000, for example 7,000 to 9,000. For example, the PEG may be, or comprise PEG 3,350, PEG 4,000 or PEG 8,000 as defined in national or regional pharmacopoeias. Optionally, the PEG used in formulations disclosed herein may comprise two or more different PEG components. Optionally, the PEG used in formulations may have at least two differing average molecular weights. PEG of the relevant molecular weights in a form suitable for use in humans is available commercially.

Examples of other osmotic agents include, but are not limited to, Pharmaburst, sorbitol, mannitol, PEG 400, PEG 4000, PEG 6000, propylene glycol, xylitol, sodium phosphate tribasic, mannitol-lactose, sodium bicarbonate, and sodium carbonate. In general, when present in the formulation or composition, the osmotic agent may be present at a concentration between 0.5-90% w/w, 2-75% w/w, typically 5-50% w/w, typically 10-40% w/w, typically 15-35% w/w.

In accordance with certain embodiments, the formulations may include the following ingredients by weight percent as set forth in Tables 1 and 2:

TABLE 1

| | First Exemplary | Second Exemplary | Third Exemplary |
|---|---|---|---|
| Ingredients | Range | Range | Range |
| Active | 2.5-50 | 5-40 | 10-30 |
| Lipids | 1-90 | 7-65 | 8-60 |
| Surfactants | 5-50 | 5-30 | 10-25 |
| Osmotic | 0-50 | 10-40 | 15-35 |
| Agent | | | |
| Other | As needed | As needed | As needed |

TABLE 2

| | First Exemplary | Second Exemplary | Third Exemplary |
|---|---|---|---|
| Ingredients | Range | Range | Range |
| THC | 1.25-25 | 2.5-20 | 5-15 |
| CBD | 1.25-25 | 2.5-20 | 5-15 |
| MCT Oil | 5-50 | 10-40 | 15-30 |
| Non-ionic | 5-50 | 5-30 | 10-25 |
| Surfactants | | | |
| Osmotic | 0-50 | 10-40 | 15-35 |
| Agent | | | |

### Pharmaceutical Compositions

This invention also provides a pharmaceutical composition comprising at least one of the formulations as described herein.

The phrase “pharmaceutically-acceptable carrier” as used herein means a pharmaceutically-acceptable material, composition or vehicle, such as a liquid or solid filler, diluent, excipient, solvent or encapsulating material, involved in carrying or transporting the subject pharmaceutical agent from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as butylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations.

Wetting agents, emulsifiers and lubricants, such as sodium lauryl sulfate, magnesium stearate, and polyethylene oxide-polybutylene oxide copolymer as well as coloring agents, release agents, coating agents, sweetening, flavoring and perfuming agents, preservatives and antioxidants can also be present in the compositions.

Formulations of the present invention include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated and the particular mode of administration. The amount of active ingredient, which can be combined with a carrier material to produce a single dosage form will generally be that amount of the compound which produces a therapeutic effect. Generally, out of 100%, this amount will range from about 1% to about 99% of active ingredient, preferably from about 0.5% to about 70%, most preferably from about 0.5% to about 40%.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules and the like), the active ingredient may be mixed with one or more pharmaceutically-acceptable carriers, such as sodium citrate or dicalcium phosphate, and/or any of the following including but not limiting to: fillers or extenders, such as starches, lactose, sucrose, glucose, mannitol, and/or silicic acid; binders, such as, for example, carboxymethylcellulose, alginates, gelatin, polyvinyl pyrrolidone, sucrose and/or acacia; humectants, such as glycerol; disintegrating agents, such as agar-agar, calcium carbonate, potato or tapioca starch, alginic acid, certain silicates, sodium carbonate, and sodium starch glycolate; solution retarding agents, such as paraffin; absorption accelerators, such as quaternary ammonium compounds; wetting agents, such as, for example, cetyl alcohol, glycerol monostearate, and polyethylene oxide-polybutylene oxide copolymer; absorbents, such as kaolin and bentonite clay; lubricants, such a talc, calcium stearate, magnesium stearate, solid polyethylene glycols, sodium lauryl sulfate, and mixtures thereof; and coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxybutylmethyl cellulose, etc), lubricant, inert diluent, preservative, disintegrant (for example, sodium starch glycolate or cross-linked sodium carboxymethyl cellulose etc), surface-active or dispersing agent. Molded tablets, may be, made by molding in a suitable machine a mixture of the drug using suitable excipients and binding processes.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxybutylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions, which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples are embedding compositions, which can be used include polymeric substances and waxes. The active ingredient can also be in micro-encapsulated form with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isobutyl alcohol, ethyl carbonate, ethyl acetate, benzyl alcohol, benzyl benzoate, butylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof. Additionally, cyclodextrins, e.g., hydroxybutyl-β-cyclodextrin, may be used to solubilize compounds.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the invention for rectal or vaginal administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active pharmaceutical agents of the invention.

Formulations of the present invention which are suitable for vaginal administration also include pessaries, tampons, creams, gels, pastes, foams or spray formulations containing such carriers as are known in the art.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically-acceptable carrier, and with any preservatives, buffers, or excipients which may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary butellants, such as chlorofluorohydrocarbons and volatile unsubstituted hydrocarbons.

Transdermal patches have the added advantage of providing controlled delivery of a compound of the present invention to the body. Such dosage forms can be made by dissolving or dispersing the pharmaceutical agents in the appropriate medium. Absorption enhancers can also be used to increase the flux of the pharmaceutical agents of the invention across the skin. The rate of such flux can be controlled, by either providing a rate controlling membrane or dispersing the compound in a polymer matrix or gel.

When the compounds of the present invention are administered as pharmaceuticals, to humans and animals, they can be given per se or as a pharmaceutical composition containing, for example, 0.1% to 99.5% (more preferably, 0.5% to 90%) of active ingredient in combination with a pharmaceutically acceptable carrier.

The compounds and pharmaceutical compositions of the present invention can be employed in combination therapies, that is, the compounds and pharmaceutical compositions can be administered concurrently with, prior to, or subsequent to, one or more other desired therapeutics or medical procedures. The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, the compound of the present invention may be administered concurrently with another agent used for reducing side effects associated with chemotherapy or radiation treatment, alleviating pain or suppressing appetite in a subject), or they may achieve different effects (e.g., control of any adverse effects).

The compounds of the invention may be administered intravenously, intramuscularly, intraperitoneally, subcutaneously, topically, transdermally, orally, or by other acceptable means. The compounds may be used to treat conditions in mammals (i.e., humans, livestock, and domestic animals), birds, lizards, and any other organism, which can tolerate the compounds.

The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

In accordance with certain aspects, the formulations and pharmaceutical compositions disclosed herein are room temperature stable for at least three months, at least six months and in some cases up to 24 months. Stable formulations are characterized by dissolution and potency as determined in accordance with test method compliant with USP and other pharmacopeial standards. In accordance with some embodiments, the formulations disclosed herein exhibit no more than a 15%, more particularly no more than a 10%, and in some cases no more than a 5% drop in potency and/or dissolution from time zero. Stability studies may be conducted in compliance with those techniques or guidelines typically used such as ICH Q6A, ICH Q6B and ICH Q1E. Techniques that may be employed to improve stability include, but are not limted to, modification of the formulation, use of dessicants in the storage container and reduced headspace in the primary and secondary container as well as in the individual capsules. The desiccant is not particularly limited. Those desiccants commonly used in the pharmaceutical industry are particularly useful. One example of a useful desiccant is silica gel. The desiccant can be supplied in the form of a sachet, cartridge or canister. A canister of silica gel can be particularly useful to improve storage stability of the formulation in a container.

### EQUIVALENTS

The representative examples which follow are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples which follow and the references to the scientific and patent literature cited herein. It should further be appreciated that the contents of those cited references are incorporated herein by reference to help illustrate the state of the art. The following examples contain important additional information, exemplification and guidance which can be adapted to the practice of this invention in its various embodiments and equivalents thereof.

### EXAMPLES

In the following non-limiting examples, all values are based on weight percent unless noted otherwise. The batches disclosed below were typically produced using a 3-step process: Manufacturing, Encapsulation and Packaging. Manufacturing includes the melting and mixing of inactive ingredients and active, under controlled temperature and continuous stirring. This is followed by dispensing pre-calculated volume of the prepared mixture solution into capsules at a reduced temperature that enables quick solidification of the solution in the capsule shell. The encapsulated product is then packaged into a pharmaceutically acceptable packaging format. Examples of suitable packaging includes, but are not limited to, polyetheylene (e.g., LDPE, HDPE) and PET, blister packaging, with silica canisters and/or nitrogen flushing.

Dispersibility tests were conducted by adding 1 ml of formulation to 500 ml of DI water at 37±0.5° C. in a glass beaker, and mixed at medium vigorous speed. In-vitro performance of formulation was assessed by visually observing the solution in the beaker against light and it was graded as per the following system:

Grade A: Rapidly forming (within 1 minute) nano emulsion, having a clear or bluish appearance.

Grade B: Rapidly forming, slightly less clear emulsion, having a bluish white appearance.

Grade C: Fine milky emulsion formed within 2 minutes.

Grade D: Dull, grayish white emulsion having slightly oily appearance that is slow to emulsify (longer than 2 minutes).

Grade E: Formulation exhibiting either poor or minimal emulsification with large oil globules present on the surface.

Various formulations were prepared containing a different solubilizing base in each formulation for incorporating the actives into liposomal suspensions. Details are presented in Table 3 below.

TABLE 3

| Ingredients | Example 1 | Example 2 | Example 3 |
|---|---|---|---|
| THC | 1.16 | 2.33 | 1.14 |
| CBD | 1.16 | 2.33 | 1.14 |
| Grape seed oil | 80.14 | n/a | 4.89 |
| Vitamin E TPGS | n/a | 43 | 34.22 |
| PEG 300 | n/a | 38.22 | 48.89 |
| Kolliphor RH 40 | 7.82 | 9.56 | n/a |
| Phospholipon 85G | 7.82 | n/a | n/a |
| Orange oil | 1.95 | 4.78 | n/a |
| Dispersibility | Grade B | Grade A | Grade B |
| Analysis | | | |

Table 4 provides some examples of additional formulations.

TABLE 4

| Ingredients | Example 4 | Example 5 | Example 6 |
|---|---|---|---|
| THC | 0.5 | 0.5 | 0.5 |
| CBD | 0.5 | 0.5 | 0.5 |
| PEG 300 | 63 | 62.84 | 63 |
| Kolliphor P407 | 12 | 12 | 12 |
| Phospholipon 85G | 12 | 12 | 12 |
| MCT oil | 10 | 10 | 10 |
| Orange oil | 2 | 2 | n/a |
| Methyl paraben | n/a | 0.1 | n/a |
| Propyl paraben | n/a | 0.01 | n/a |
| BHT | n/a | 0.05 | n/a |
| Peppermint oil | n/a | n/a | 2 |
| Dispersibility Analysis | Grade B | Grade B | Grade B |

The following formulations were prepared and tested for use as self-emulsifying drug delivery systems (SEDDS). The weights provided in the table refer to mg/capsule.

TABLE 5

| | Example | Example | Example | Example | Example | Example |
|---|---|---|---|---|---|---|
| Ingredients | 7 | 8 | 9 | 10 | 11 | 12 |
| THC | — | 2.5 | 2.5 | 2.5 | 2.5 | — |
| CBD | 200 | 2.5 | 2.5 | 2.5 | 2.5 | 200 |
| PEG 300 | 160 | 315 | — | — | 315 | 160 |
| Gelucire 48/16 | — | — | — | 415 | — | — |
| Kolliphor P407 | 60 | — | — | — | 60 | 60 |
| Kolliphor RH40 | — | 60 | 100 | 30 | — | — |
| Phospholipon | 40 | 60 | — | — | 60 | 40 |
| 85G | | | | | | |
| MCT oil | 30 | 50 | 50 | 50 | 50 | 30 |
| Vitamin E TPGS | — | — | 345 | — | — | — |
| Peppermint oil | 10 | 10 | — | — | 10 | 10 |
| Dispersibility | Grade B | Grade B | Grade C | Grade B | Grade B | Grade B |
| Analysis | | | | | | |

The following formulations were prepared and tested for dispersibility and dissolution.

TABLE 6

| Ingredients | Example 13 | Example 14 | Example 14 | Example 15 |
|---|---|---|---|---|
| THC | 2.5 | 10 | 2.5 | 10 |
| MCT oil | 32.5 | 30 | 18.17 | 22.5 |
| Gelucire 48/16 | 32.5 | 30 | 39.7 | 27.5 |
| Kolliphor EL | 32.5 | 30 | 39.7 | 22.5 |
| PEG 3350 | — | — | — | 17.5 |
| Dispersibility | Grade A | Grade A | Grade A | Grade A |
| Analysis | | | | |
| Complete | No release | No release | after | Within 20 |
| Dissolution | within | within | 1.5 hour | minutes |
| | an hour | an hour | | |

The following oral fast acting capsule formulations were prepared and tested for dispersibility and dissolution.

TABLE 7

| | Example | Example | Example | Example | Example | Example | Example |
|---|---|---|---|---|---|---|---|
| Ingredients | 16 | 17 | 18 | 19 | 20 | 21 | 22 |
| THC | 10 | 10 | 2.5 | 0 | 0 | 0 | 40 |
| CBD | 0 | 0 | 2.5 | 40 | 40 | 40 | — |
| MCT oil | 22.5 | 30 | 23.75 | 15 | 17.5 | 10 | 14 |
| Gelucire | 27.5 | 20 | 29.11 | 18.38 | 10 | 25 | 14 |
| 48/16 | | | | | | | |
| Kolliphor | 22.5 | 20 | 23.75 | 15 | 15 | 15 | 22 |
| EL | | | | | | | |
| PEG 3350 | 17.5 | 20 | 18.39 | 11.62 | 17.5 | 10 | 10 |
| Dispersibility | Grade A | Grade A | Grade A | Grade A | Grade A | Grade A | Grade A |
| Analysis | dispersion | Dispersion | dispersion | Dispersion | Dispersion | Dispersion | Dispersion |

Additional examples are provided below in Table 8 (all values in % wt/wt with DI water accounting for any residual amount):

TABLE 8

| Ingredients | Example 23 | Example 24 |
|---|---|---|
| THC | 2.5 | 10 |
| CBD | 2.5 | 10 |
| MCT Oil | 23.75 | 19 |
| Gelucire 48/16 | 29.11 | 19 |
| Kolliphor EL | 23.75 | 27 |
| PEG 4000 | 18.39 | 15 |
| Dispersibility | Grade A, Water soluble, | Grade A, Water soluble, |
| Analysis | Clear solution | Clear solution |

### Stability Testing

A single 70 g batch of Example 24 was prepared to use in the following analysis. The composition was encapsulated and packaged as described below (Table 9—packaging summary), with 10 capsules per bottle. The samples (one bottle each) were evaluated for stability over time (0 to 3 months) and under two different test conditions (40° C./75% RH and 25° C./60% RH).

TABLE 9

| | | | | | Silica Dessicant |
|---|---|---|---|---|---|
| Formula | Bottle | | Capsule | Fill volume | Canister? |
| F1 | 100 | ml PET | Size 2 | 0.1 ml | No |
| F2 | 100 | ml PET | Size 2 | 0.1 ml | Yes |
| F3 | 60 | ml HDPE | Size 2 | 0.1 ml | No |
| F4 | 100 | ml PET | Size 3 | 0.1 ml | No |

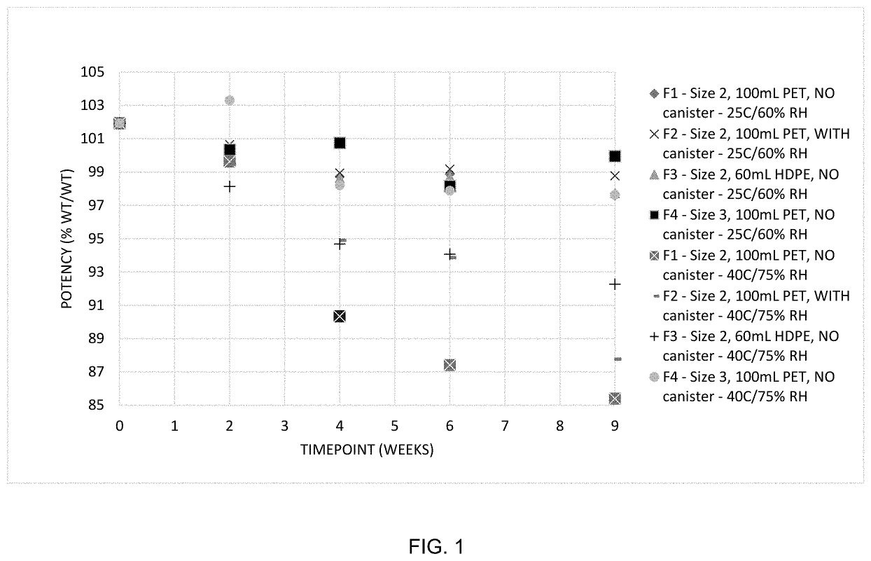

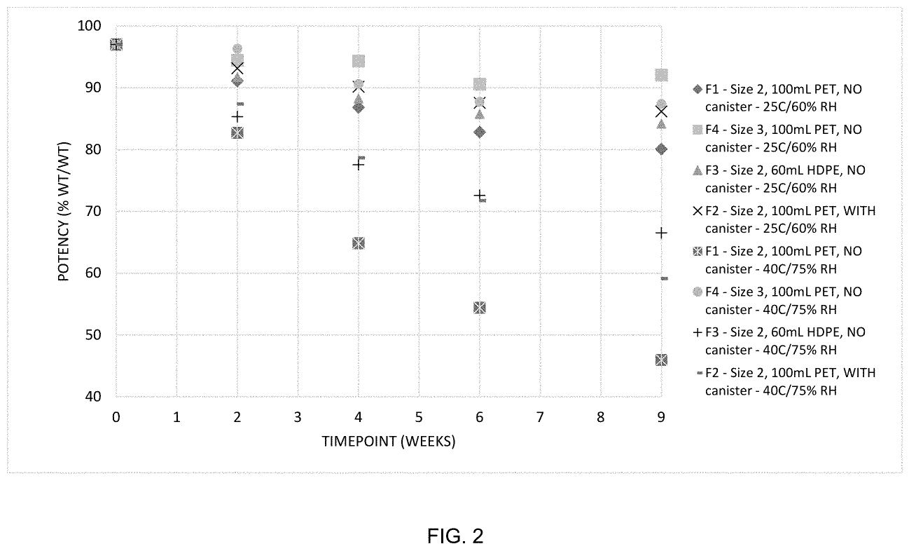

At T₀, 2 week, 4 week, 6 week, and 9 week time points, the potencies of the capsules were evaluated by measuring concentration of the APIs (THC and CBD) by HPLC-UV and calculating potency as a percentage based on measured weight/label weight. Results are provided below in Tables 10 and 11 and in FIGS. 1 and 2.

CBD and THC potency in the disclosed capsules for four formulae at two storage conditions over a period of 9 weeks.

TABLE 10
CBD potency in capsules (% wt/wt)

| | 0 wk | 2 wk | 4 wk | 6 wk | 9 wk |
|---|---|---|---|---|---|
| F1 - Size 2, 100 mL PET, | 101.93 | 99.69 | 98.72 | 98.87 | 97.58 |
| NO canister - 25 C./60% RH | | | | | |
| F2 - Size 2, 100 mL PET, | 101.93 | 100.64 | 98.95 | 99.18 | 98.78 |
| WITH canister - 25 C./ | | | | | |
| 60% RH | | | | | |
| F3 - Size 2, 60 mL HDPE, | 101.93 | 100.16 | 98.63 | 98.71 | 97.72 |
| NO canister - 25 C./60% RH | | | | | |
| F4 - Size 3, 100 mL PET, | 101.93 | 100.35 | 100.75 | 98.16 | 99.95 |
| NO canister - 25 C./60% RH | | | | | |
| F1 - Size 2, 100 mL PET, | 101.93 | 99.63 | 90.33 | 87.41 | 85.39 |
| NO canister - 40 C./75% RH | | | | | |
| F2 - Size 2, 100 mL PET, | 101.93 | 99.48 | 94.90 | 93.86 | 87.77 |
| WITH canister - 40 C./ | | | | | |
| 75% RH | | | | | |
| F3 - Size 2, 60 mL HDPE, | 101.93 | 98.14 | 94.67 | 94.06 | 92.27 |
| NO canister - 40 C./75% RH | | | | | |
| F4 - Size 3, 100 mL PET, | 101.93 | 103.31 | 98.21 | 97.88 | 97.64 |
| NO canister - 40 C./75% RH | | | | | |

TABLE 11
THC potency in capsules (% wt/wt)

| | 0 wk | 2 wk | 4 wk | 6 wk | 9 wk |
|---|---|---|---|---|---|
| F1 - Size 2, 100 mL PET, | 97.00 | 91.12 | 86.82 | 82.83 | 80.06 |
| NO canister - 25 C./60% RH | | | | | |
| F2 - Size 2, 100 mL PET, | 97.00 | 93.16 | 90.16 | 87.57 | 86.18 |
| WITH canister - 25 C./60% RH | | | | | |
| F3 - Size 2, 60 mL HDPE, | 97.00 | 91.73 | 88.23 | 85.74 | 84.19 |
| NO canister - 25 C./60% RH | | | | | |
| F4 - Size 3, 100 mL PET, | 97.00 | 94.49 | 94.34 | 90.60 | 92.07 |
| NO canister - 25 C./60% RH | | | | | |
| F1 - Size 2, 100 mL PET, | 97.00 | 82.71 | 64.85 | 54.39 | 45.96 |
| NO canister - 40 C./75% RH | | | | | |
| F2 - Size 2, 100 mL PET, | 97.00 | 87.37 | 78.65 | 71.73 | 59.12 |
| WITH canister - 40 C./75% RH | | | | | |
| F3 - Size 2, 60 mL HDPE, | 97.00 | 85.32 | 77.54 | 72.58 | 66.50 |
| NO canister - 40 C./75% RH | | | | | |
| F4 - Size 3, 100 mL PET, | 97.00 | 96.32 | 90.63 | 87.73 | 87.40 |
| NO canister - 40 C./75% RH | | | | | |

Results for F1 compared to F2 show that both THC and CBD potency is protected by addition of the silica canister under both storage conditions. This effect is much more apparent at the high temperature/high humidity storage conditions (40° C./75% RH), where over 9 weeks, without the canister (F1), CBD concentration drops from 101.93% to 85.39% and THC concentration drops from 97.00% to 45.96% (FIG. 1). With addition of the silica canister (F2), CBD concentration drops from 101.93% to 87.77% and THC concentration drops from 97.00% to 59.12% (FIG. 2). Here, addition of the silica canister may be very effective in improving stability in high-humidity, warm climates and has minor protective capacity at moderate temperature/humidity (25° C./60% RH).

Results for F1 compared to F3 show that stability of the API can vary depending on the bottle size and material. At moderate temperature/humidity (25° C./60% RH), there is minimal effect on API stability with the bottle variation. However, at the high temperature/high humidity storage conditions (40° C./75% RH), this effect is much more apparent, especially for THC. At these conditions over 9 weeks, in the 100 mL PET bottle (F1), CBD concentration drops from 101.93% to 85.39% and THC concentration drops from 97.00% to 45.96% (FIG. 1). In the 60 mL HDPE bottle (F3), CBD concentration drops from 101.93% to 92.27% and THC concentration drops from 97.00% to 66.50% (FIG. 2). Therefore, the use of a 60 mL HDPE bottle may be very effective in improving stability in high-humidity, warm climates.

Comparing results of F1 to F4 shows the effect of capsule size (and thus headspace inside capsule) on API stability. Reduced headspace provides significant improvement in protective effects on API stability—in both compounds at all storage conditions evaluated. The high temperature/high humidity storage conditions (40° C./75% RH) may represent accelerated life conditions and the largest protective effects were observed in this group of samples. At these conditions over 9 weeks, in the larger size 2 capsules (F1), CBD concentration drops from 101.93% to 85.39% and THC concentration drops from 97.00% to 45.96% (FIG. 1). In the smaller size 3 capsules (F4), CBD concentration only drops from 101.93% to 97.64% and THC concentration only drops from 97.00% to 87.40% (FIG. 2). At moderate temperature/humidity (25° C./60% RH), both THC and CBD are within 10% of the T₀ concentrations of both CBD and THC at 9 weeks.

Overall, an improvement in API stability was obtained with any of these encapsulation and packaging changes, however capsule headspace has the most significant impact.

Table 12 provides additional examples, including some consumables.

TABLE 12

| | | | | | | | Example | Example |
|---|---|---|---|---|---|---|---|---|
| | Example | Example | Example | Example | Example | Example | 31 | 32 |
| Ingredients | 25 | 26 | 27 | 28 | 29 | 30 | Beverage Base | |
| THC | 0.1 | | | | | | 0.1 | 0.0 |
| Walnut Oil | 0.7 | 0.7 | 2.5 | 0.7 | 0.7 | 0.3 | 0.0 | 0.000 |
| MCT oil | 0.7 | 0.7 | 2.5 | 0.7 | 0.7 | 0.3 | 0.3 | 0.004 |
| Lecithin | 1.4 | 1.4 | 5.0 | 1.4 | 0.7 | 0.3 | 0.3 | 0.001 |
| Vitamin E | 0.0 | 0.0 | 0.0 | 0.3 | 0.3 | 0.1 | 0.1 | 0.001 |
| TPGS | | | | | | | | |
| Ascorbic Acid | 0.3 | 0.0 | 0.0 | 0.0 | 0.0 | 0.0 | 0.0 | 0.000 |
| Water | 25.4 | 25.7 | 10.0 | 25.4 | 26.1 | 0.0 | 5.0 | 5.7 |

Additional examples are provided below in Table 13:

TABLE 13

| | Example 33 | Example 34 | Example 35 | Example 36 |
|---|---|---|---|---|
| Ingredients | (F5) | (F6) | (F7) | (F8) |
| THC | 2.5 | 2.5 | 2.5 | 2.5 |
| CBD | 2.5 | 2.5 | 2.5 | 2.5 |
| Gelucire 48/16 | 22.75 | 22.75 | 22.75 | 22.75 |
| MCT Oil | 22.75 | 22.75 | 22.75 | 22.75 |
| PEG 4000 | 18.75 | 18.75 | 18.75 | 18.75 |
| Kolliphor EL | 30.75 | 30.75 | 0 | 0 |
| Vitamin E | 0 | 0 | 30.75 | 30.75 |
| TPGS | | | | |
| | 100 mg/cap | 400 mg/cap | 100 mg/cap | 400 mg/cap |
| | into size | into size | into size | into size |
| | 3 capsules | 1 capsules | 3 capsules | 1 capsules |

The examples in Table 13 were prepared in accordance with the following process. The ingredients were added in the following order: Gelucire 48/16 (melted at 55° C.); PEG 4000; Kolliphor EL or VitE TPGS (melted); MCT oil; THC; CBD. After each ingredient addition and prior to addition of the next ingredient, the composition was mixed for 10 minutes at 55° C. and 400 RPM. The final mixture was homogenized at 25,000 RPM for 3 minutes. Capsules were filled while the final mixture was stirred at 70 RPM and maintained at 55° C. The 400 mg/cap formulations were filled into size 1 capsules and the 100 mg/cap formulations were filled into size 3 capsules.

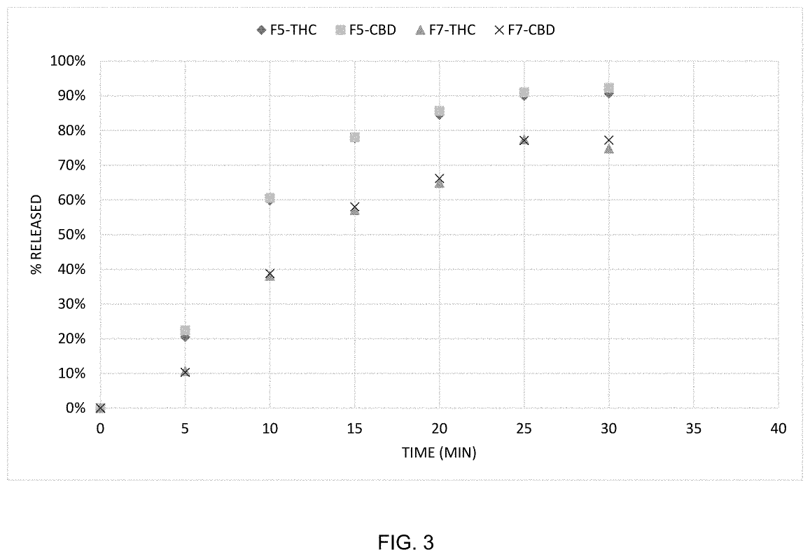

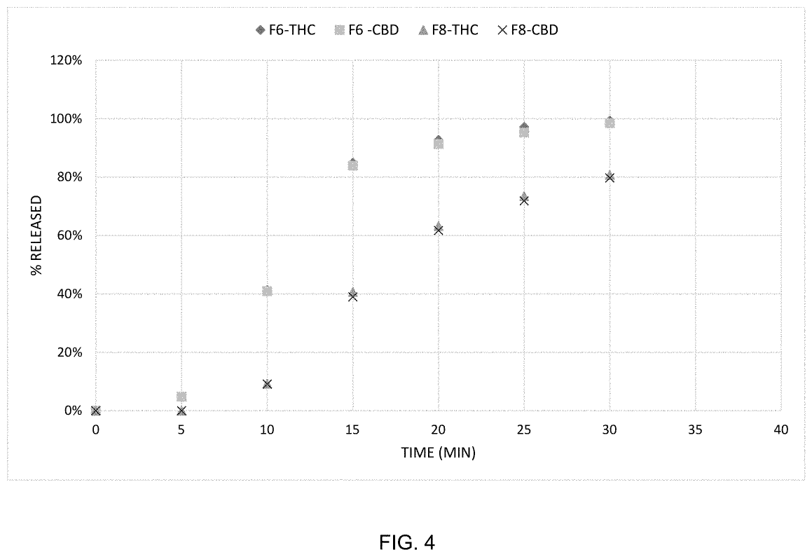

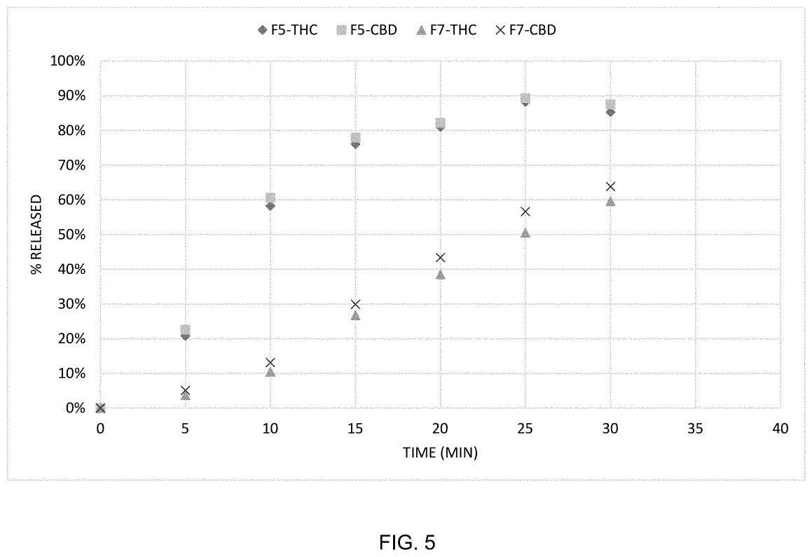

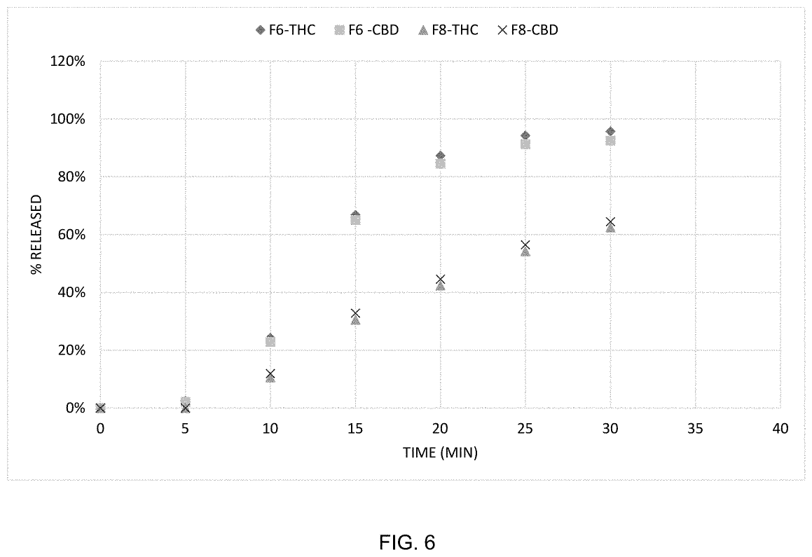

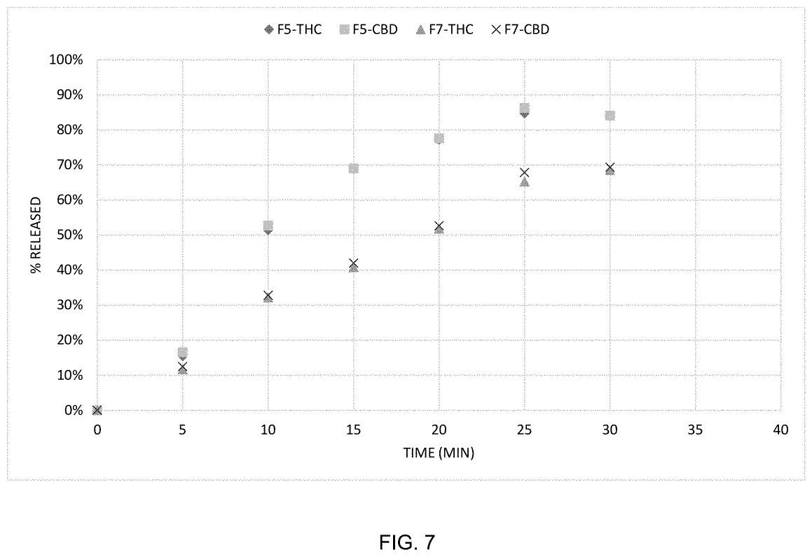

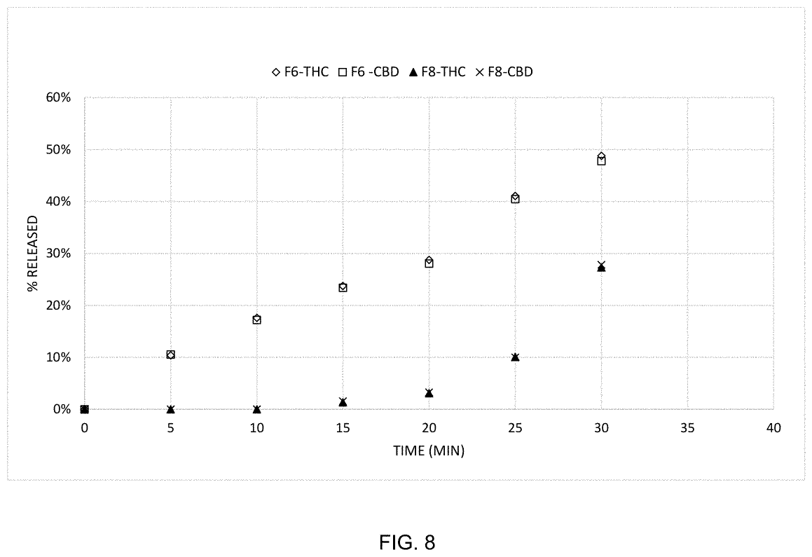

Formulations F5-F8 (Examples 33-36, respectively) were subjected to in-vitro dissolution studies in three media: a) Water b) Simulated Gastric Fluid and c) Simulated Intestinal Fluid. Results are provided in FIGS. 3-8, which show results for various examples in water (FIGS. 3 and 4), simulated gastric fluid (FIGS. 5 and 6) and simulated intestinal fluid (FIGS. 7 and 8). Based on the in-vitro dissolution graphs shown in FIGS. 3-8, the formulations containing Kolliphor EL are not significantly influenced by media and continues to demonstrate an immediate release of drug from the dosage format. Vitamin E TPGS is beneficial in water and potentially in simulated fluid however in intestinal fluid the formulations appear to be less soluble and exhibits a substantial lag in drug release.

Although the invention has been described and illustrated in the foregoing illustrative embodiments, it is understood that the present disclosure has been made only by way of example, and that numerous changes in the details of implementation of the invention can be made without departing from the spirit and scope of the invention, which is limited only by the claims that follow. Features of the disclosed embodiments can be combined and/or rearranged in various ways within the scope and spirit of the invention to produce further embodiments that are also within the scope of the invention.

1. A formulation comprising:
a cannabinoid or cannabinoid analogue in a delivery system, wherein the delivery system comprises: a mixture of at least one lipid and at least one surfactant, wherein the formulation self-emulsifies in an aqueous medium to produce a plurality of particles having a mean particle size of about 1 to about 150 nm.

2. The formulation of claim 1, wherein the cannabinoid or cannabinoid analogue comprises at least one of delta-9-tetrahydrocannabinol [THC] and cannabidiol [CBD].

3. The formulation of claim 1, wherein the delivery system further comprises an osmotic agent.

4. The formulation of claim 3, wherein the osmotic agent comprises polyethylene glycol having an average molecular weight of about 300 to 6,000 Daltons.

5. The formulation of claim 4, wherein the osmotic agent comprises polyethylene glycol having an average molecular weight of about 1,000 to 5,000 Daltons.

6. The formulation of claim 5, wherein the osmotic agent comprises polyethylene glycol having an average molecular weight of about 2,500 to 4,500 Daltons.

7. The formulation of claim 1, wherein the delivery system further comprises a co-solvent.

8. The formulation of claim 7, wherein the co-solvent is water-soluble.

9. The formulation of claim 1, wherein the cannabinoid or cannabinoid analogue is present in the formulation in an amount of from about 1 to 60% based on weight.

10. The formulation of claim 1, wherein the lipid(s) are present in the formulation in an amount of from about 1% to 90% based on weight.

11. The formulation of claim 1, wherein the surfactant(s) are present in the formulation in an amount of from about 0.01% to 45% based on weight.

12. The formulation of claim 1, wherein the lipid is selected from the group consisting of medium-chain triglycerides, phospholipids, phospholipid derivatives, vitamin E derivatives, glyceryl dibehanate, behenoyl polyoxyl-8-glycerides, soybean oil glyceryl palmitostearate, glyceryl monostearate PEG-75 stearate and combinations thereof.

13. The formulation of claim 1, wherein the surfactant is selected from the group consisting of polyethoxylated castor oil, poloxamers, hydrogenated castor oil ethoxylates, polyoxylethylene stearates, polyoxyl glycerides, glycol monolaureate, polyglyceryl dioleate and combinations thereof.

14. The formulation of claim 13, wherein the polyethoxylated castor oil comprises polyoxyl 35 castor oil.

15. A pharmaceutical composition comprising the formulation of claim 1, wherein the pharmaceutical composition is in the form of a liquid dosage form, a solid dosage form, a capsule, a semi-solid, an orodispersible film, beverage, food or orodispersible tablet.

16. The pharmaceutical composition of claim 15, wherein the pharmaceutical composition is stable for a period of at least six months at room temperature as measured in accordance with ICH Guidance.

17. A method of reducing side effects associated with chemotherapy or radiation treatment, alleviating pain or suppressing appetite in a subject in need thereof comprising administering to the subject the pharmaceutical composition of claim 15.
